(19) United States
(12) Patent Application Publication
(10) Pub. No.: US 2009/0099062 A1
(43) Pub. Date: Apr. 16, 2009

(54) Pyrvinium For The Treatment of Cancer

(76) Inventors: Ethan Lee, Brentwood, TN (US); Laura Lee, Brentwood, TN (US); Curtis Thorne, Nashville, TN (US); Emilios Tahinci, Nashville, TN (US); Kelly Christian Meyers, Nashville, TN (US)

Correspondence Address: FULBRIGHT & JAWORSKI L.L.P., 600 CONGRESS AVE., SUITE 2400, AUSTIN, TX, 78701, US

(21) Appl. No.: 12/129,250

(22) Filed: May 29, 2008

Related U.S. Application Data
(60) Provisional application No. 60/941,205, filed on May 31, 2007.

(51) Int. Cl. A61K 38/02; G01N 33/48; C12Q 1/66; C12N 5/06; A61K 31/337

(52) U.S. Cl. 514/2; 436/63; 435/8; 435/375; 514/449

(57) ABSTRACT

The present invention concerns a pyrvinium compound or an analog thereof for the treatment of cancers. This compound inhibits Wnt activity in the cells of cancers such as adrenocortical, hepatocellular, hepatoblastoma, malignant melanoma, ovarian, Wilm's tumor, Barrett's esophageal, glioma, bladder, breast, gastric, head & neck, lung cell, mesothelioma, and cervical cancers. The present invention also provides a method for assaying for compounds that alter Wnt pathway activity. Also provided are methods for treating Wnt-related non-cancer disease states.

This application claims benefit of priority to U.S. Provisional Application Ser. No. 60/941,205, filed May 31, 2007, the entire contents of which are hereby incorporated by reference.

The government owns rights in the present invention pursuant to grant numbers CA56704, CA68485 and 5P30AR41943 each from the National Institutes of Health.

### BACKGROUND OF THE INVENTION

1. Field of the Invention

The present invention relates generally to the fields of cancer biology and cancer therapeutics. More particularly, it concerns the use of a pyrvinium compound or salts or analogs thereof, in the treatment of cancer, particularly colon and breast cancer.

2. Description of Related Art

Inappropriate activation of the Wnt pathway is believed to be the initial event leading to colorectal cancer in over 85% of all sporadic cases in the Western world. Furthermore, Wnt signaling is thought to contribute to proliferation of breast cancer, as well as a number of other cancers. As such, it is a therapeutic target that is of great interest to the field.

However, to date there are limited reports of biochemical screens that examine the effect of candidate inhibitor substances on Wnt signaling, despite the fact that they would be expected to be potent inhibitors of colon and breast cancer, as well as numerous other cancers and other disease. Thus, there remains a need in the field for such assays, for the drugs identified therewith, and for therapies utilizing such drugs.

### SUMMARY OF THE INVENTION

As stated above, there remains a need in the art for effective therapeutic agents for treating and/or prevention of cancers such as colon and lung cancer. Such therapeutic agents may include the use of synthetic small molecules that target molecules involve in the initiation and/or progression of a cancer and exhibit minimal toxicity to normal human cells. The present invention is therefore directed to a cancer therapeutic for the treatment and/or prevention of cancer that overcomes the toxicity, side effects or resistance offered by current chemotherapeutic agents.

In one embodiment, the present invention provides a method of identifying modulators for the Wnt pathway comprising (a) providing a Xenopus laevis egg extract comprising axin and β-catenin; (b) contacting the extract with a candidate substance; (c) assessing the degradation and/or stability of the axin and/or β-catenin, wherein a change in the degradation and/or stability of axin and/or β-catenin, as compared to the degradation and/or stability of axin and/or β-catenin in the absence of the candidate substance, indicates that the candidate substance is a modulator of the Wnt pathway. The axin and/or β-catenin molecules may be labeled, such as by fusion with a fluorescent protein (e.g., luciferase, GPF, CFP, YFP, or ECPF). Alternatively, labeling may be with a radioactive label or a dye.

The modulator may increase degradation of β-catenin and increase stability of axin, and the modulator may be an inhibitor of the Wnt pathway. The modulator may increase degradation of axin and increase stability of β-catenin, and the modulator is an activator of the Wnt pathway. The candidate substance is a organopharmaceutical drug, an oligonucleotide or polynucleotide, a peptide or polypeptide.

In another embodiment, there is provided a method of inhibiting a cancer cell selected from the group consisting of an adrenocortical cancer cell, a hepatocellular cancer cell, a hepatoblastoma cell, a malignant melanoma cell, a ovarian cancer cell, a Wilm's tumor cell, a Barrett's esophageal cancer cell, a bladder cancer cell, a breast cancer cell, a gastric cancer cell, a head & neck cancer cell, a lung cancer cell, a mesothelioma cell, a cervical cancer cell, a uterine cancer cell, a myeloid leukemia cancer cell, a lymphoid leukemia cancer cell, a pilometricoma cancer cell, a medulloblastoma cancer cell, a glioblastoma cell, and a familial adenomatous polyposis cancer cell comprising contacting the cancer cell with a composition comprising pyrvinium. The cancer cell may be located in a subject, such as a human subject. The cancer cell may be metastatic, multi-drug resistant, or recurrent. The cancer cell may has a mutation in APC, β-catenin, and/or axin. The pyrvinium composition may be contacted with the cancer cell more than once. Inhibiting may comprise reducing cancer cell growth or killing the cancer cell. The pyrvinium composition may be administered orally, intravenously, intratumorally, into tumor vasculature, or regional to the tumor.

The method may further comprise subjecting the cancer cell with a second anti-cancer therapy, such as chemotherapy, radiotherapy, gene therapy, immunotherapy, hormone therapy, toxin therapy, protein/peptide therapy, or surgery. The second anti-cancer therapy may be given prior to the pyrvinium composition, after the pyrvinium composition, or at the same time as the pyrvinium composition.

In yet another embodiment, there is provided a method of treating a non-cancer disease state have a Wnt signaling abnormality comprising administering to a subject in need thereof a composition comprising pyrvinium. The non-cancer disease may be autism, rheumatoid arthritis, schizophrenia, increased bone density, cardiac hypertrophy, Alzheimer's Disease, coronary artery disease, obesity, osteoporosis, familial exudative vitreoretinopathy, type II diabetes, pulmonary fibrosis, inflammation, or wound healing. The subject may be a human. The method may further comprise subjecting the cancer cell with a second therapy, where the second therapy is given prior to the pyrvinium composition, after the pyrvinium composition or at the same time as the pyrvinium composition. The pyrvinium composition may be administered more than once.

In still other embodiments, there are provided:
a method of stimulating a stem cell comprising contacting the stem cell with a composition comprising pyrvinium; and
a method of inhibiting a well-vascularized tumor in a subject comprising administering to the subject a composition comprising pyrvinium.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Pyrvinium inhibits LRP6-mediated degradation of Axin in Xenopus extract in a dose-dependent manner. Addition of LRP6 to Xenopus extract stimulates degradation of exogenously added Axin after 3 hr; degradation is blocked by addition of 10, 100, or 1000 mM Pyrvinium.

FIG. 2. β-catenin levels are reduced in cells treated with pyrvinium. 293 HEK cells were treated with Wnt3a or Wnt3a plus pyrivnium (1 μM) for 24 hr. Cell were lysed and Western blot analysis performed using anti-β-catenin or actin (loading control) antibodies.

FIG. 3. Pyrvinium inhibits Wnt-mediated gene transcription in mammalian cultured cells in a dose-dependent manner. 293 HEK cells stably transfected with a Topflash luciferase transcriptional reporter were incubated 24 hr with Wnt3a-conditioned media. Pyrvinium was then added to the media at indicated concentrations and harvested 24 hr later for measuring luciferase activity using the Steady Glo Assay (Promega). Experiments were performed in triplicate. Error bars are SEM.

FIG. 4. Effect of Pyrviniums on transcription of endogenous Wnt targets. HEK293 cells were stimulated with Wnt3a conditioned medium and 1 μM pyrvinium pamoate for 24 hr. Total mRNA was isolated and RT-PCR performed for the indicated transcripts. GAPDH is a loading control.

FIG. 5. Xwn8-mediated secondary axis formation in Xenopus embryos is blocked by Pyrvinium. Injection of Xwnt8 mRNA (0.5 pg) into one of the ventral bastomeres of 4-cell stage embryos induces secondary axis formation in 53% of injected embryos. In contrast, no embryos injected with 0.5 pg Xwnt8 mRNA plus 20 μM pyrvinium have duplicated axes. 15 embryos were injected for each condition.

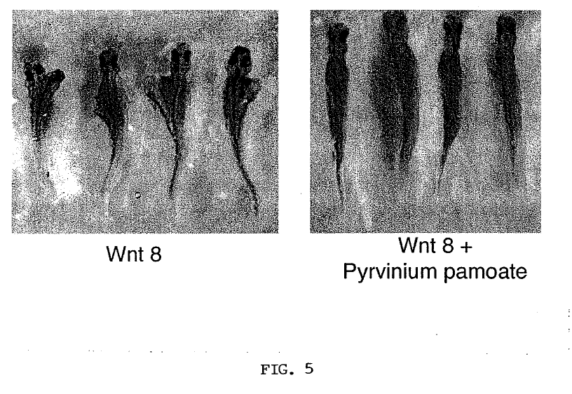

FIG. 6. The cytotoxic effect of pyrvinium on cancer cells parallels its Wnt inhibition activity. Colorectal cancer cell lines, SW620 and SW480, and a breast cancer cell line, MDA-MB-231, were incubated for 48 hr with indicated concentrations of pyrvinium. Viable cells were counted using the CellTiter-Glo assay (Promega). Experiments were performed in triplicates. Error bars are SEM.

FIG. 7. Pyrvinium decreases β-catenin levels in a colorectal cancer cell line. HCT116 cells were incubated with various concentration of pyrvinium for 24 hr. Cells were lysed in hypotonic buffer, and ctyoplasmic β-catenin levels were assessed. Tubulin is a loading control.

FIG. 8. Pyrvinium inhibits growth of the colorectal cancer cell line, HCT116, in a dose-dependent manner. HCT116 cells were incubated with indicated concentrations of pyrvinium and viable cells counted at indicated times using the CellTiter-Glo assay (Promega). Experiments were performed in triplicates. Error bars are SEM.

FIG. 9. Increased apoptosis in cells treated with WS30. Colorectal cancer cell lines, HCT116, SW620 and SW480, and the breast cancer cell line, MDA-MB-231, were incubated for 48 hr with 1 μM pyrvinium or vehicle. Caspase activity was then measured with Caspase-Glo3/7 (Promega). For each cell line, caspase activity is normalized to vehicle control. Experiments were performed in triplicate. Error bars are SEM.

FIG. 10. Pyrvinum abolishes sphere formation in primary human glioblastoma cultures. Incubations of primary cultures from three human glioblastoma lines (7030, 7081, and 7192) blocks the ability of cells to form neurospheres when plated on tumor stem cell medium containing EGF and bFGF. Right panel—phase contrast micrographs of representative neurospheres incubated for 48 hours in the absence and presence of 100 nM pyrvinium. Left panel—quantification of the number of neurospheres formed (per 200 cells plated) for the three primary glioblastoma lines.

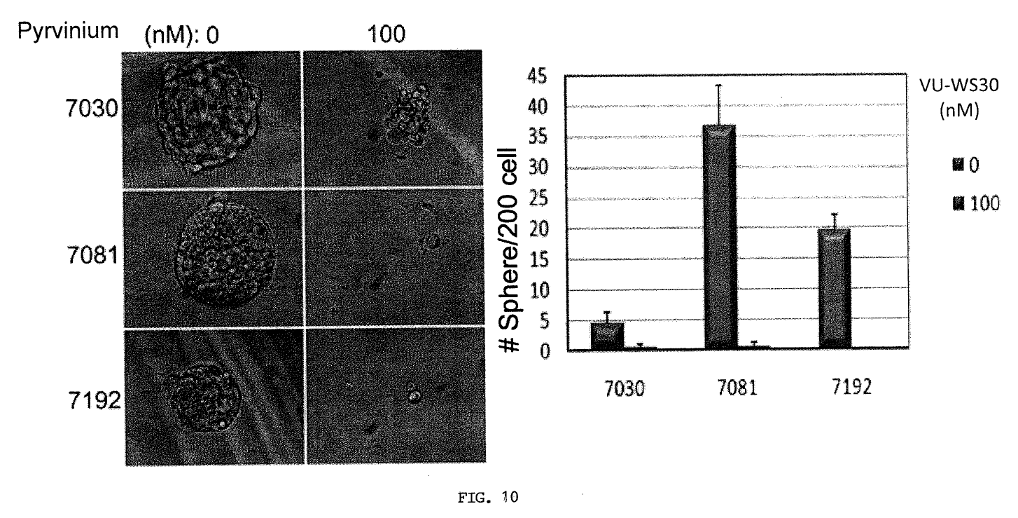

FIGS. 11A-B. Pyrvininium potentiates the cytotoxic drug, 5-FU, to promote apoptosis. (FIG. 11A) The SW620 colorectal cancer line was incubated with Vehicle, 5-FU (5 mM), pyrvinium (100 nM), or 5-FU (5 mM) plus pyrvinium (100 nM) and the morphology assessed by phase contrast microscopy. Only the 5-FU plus pyrvinium treated cells show cellular blebbing consistant with significant apoptosis. (FIG. 11B) This result is confirmed with 5-FU-plus-pyrvinium-treated cells demonstrating significant activation of the apoptotic pathway as measured by Caspase 3/7 activity

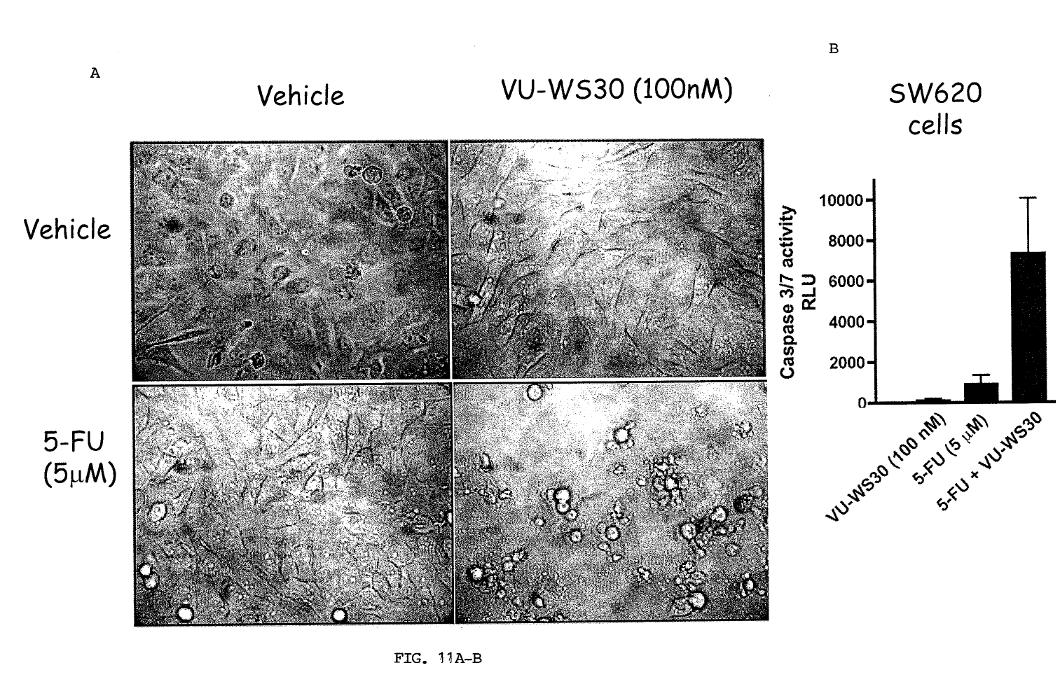

### DESCRIPTION OF ILLUSTRATIVE EMBODIMENTS

### I. The Present Invention

The present inventors have developed a biochemical screen using Xenopus laevis egg extracts to identify regulators of the canonical Wnt pathway. Normally, in the absence of Wnt signaling, the proteins β-catenin and axin are degraded and stabilized, respectively. Activation of the Wnt pathway results in stabilization of β-catenin and degradation of axin. The inventors show that this can be detected in Xenopus egg extracts by measuring β-catenin fused to firefly luciferase and axin fused to Renilla luciferase and using recombinant protein encoding the intracellular domain of the Wnt receptor, LRP6, to activate the Wnt pathway. Adapting this assay for high-throughput screening, the inventors identified both inhibitors and activators of the Wnt pathway. Of particular interest are flavonoid compounds that have the ability to inhibit both breast and cancer cells. In particular, the anti-parasitic compound pyrvinium has shown particular utility in inhibiting these types of cancer cells.

### II. Wnt Pathway Signaling

The wnt signaling pathway describes a complex network of proteins most well known for their roles in embryogenesis and cancer, but also involved in normal physiological processes in adult animals. Wnt proteins form a family of highly conserved secreted signaling molecules that regulate these interactions. Insights into the mechanisms of Wnt action have emerged from several systems: genetics in Drosophila and Caenorhabditis elegans; biochemistry in cell culture and ectopic gene expression in Xenopus embryos. Many Wnt genes in the mouse have been mutated, leading to very specific developmental defects. As currently understood, Wnt proteins bind to receptors of the Frizzled and LRP families on the cell surface. Through several cytoplasmic relay components, the signal is transduced to β-catenin, which then enters the nucleus and forms a complex with TCF to activate transcription of Wnt target genes.

The name Wnt was coined as a combination of Wg (“wingless”) and Int. The wingless gene had originally been identified as a segment polarity gene in Drosophila melanogaster that functions during embryogenesis. and also during adult limb formation during metamorphosis. The INT genes were originally identified as vertebrate genes near several integration sites of mouse mammary tumor virus (MMTV). The Int-1 gene and the wingless gene were found to be homologous, with a common evolutionary origin evidenced by similar amino acid sequences of their encoded proteins.

Mutations of the wingless gene in the fruit fly were found in wingless flies, while tumors caused by MMTV were found to have copies of the virus integrated into the genome forcing overproduction of one of several Wnt genes. The ensuing effort to understand how similar genes produce such different effects has revealed that Wnts are a major class of secreted morphogenic ligands of profound importance in establishing the pattern of development in the bodies of all multicellular organisms studied.

The Wnt pathway involves a large number of proteins that can regulate the production of Wnt signaling molecules, their interactions with receptors on target cells and the physiological responses of target cells that result from the exposure of cells to the extracellular Wnt ligands. Although the presence and strength of any given effect depends on the Wnt ligand, cell type, and organism, some components of the signaling pathway are remarkably conserved in a wide variety of organisms, from Caenorhabditis elegans to humans. Protein homology suggests that several distinct Wnt ligands were present in the common ancestor of all bilaterian life, and certain aspects of Wnt signaling are present in sponges and even in slime molds.

The canonical Wnt pathway describes a series of events that occur when Wnt proteins bind to cell-surface receptors of the Frizzled family, causing the receptors to activate Dishevelled family proteins and ultimately resulting in a change in the amount of β-catenin that reaches the nucleus. Dishevelled (DSH) is a key component of a membrane-associated Wnt receptor complex which, when activated by Wnt binding, inhibits a second complex of proteins that includes axin, GSK-3, and the protein APC. The axin/GSK-3/APC complex normally promotes the proteolytic degradation of the β-catenin intracellular signaling molecule. After this “β-catenin destruction complex” is inhibited, a pool of cytoplasmic β-catenin stabilizes, and some β-catenin is able to enter the nucleus and interact with TCF/LEF family transcription factors to promote specific gene expression. Some additional details of the pathway are described below.

Cell surface Frizzled (FRZ) proteins usually interact with a transmembrane protein called LRP. LRP binds Frizzled, Wnt and axin and may stabilize a Wnt/Frizzled/LRP/Discheveled/axin complex at the cell surface. In vertebrates, several secreted proteins have been described that can modulate Wnt signaling by either binding to Wnts or binding to a Wnt receptor protein. For example, Sclerostin can bind to LRP and inhibit Wnt signaling. The part of the pathway linking the cell surface Wnt-activated Wnt receptor complex to the prevention of β-catenin degradation is still under investigation. There is evidence that trimeric G proteins can function downstream from Frizzled. It has been suggested that Wnt-activated G proteins participate in the disassembly of the axin/GSK3 complex.

Several protein kinases and protein phosphatases have been associated with the ability of the cell surface Wnt-activated Wnt receptor complex to bind axin and disassemble the axin/GSK3 complex. Phosphorylation of the cytoplasmic domain of LRP by CK1 and GSK3 can regulate axin binding to LRP. The protein kinase activity of GSK3 appears to be important for both the formation of the membrane-associated Wnt/FRZ/LRP/DSH/Axin complex and the function of the Axin/APC/GSK3/β-catenin complex. Phosphorylation of β-catenin by GSK3 leads to the destruction of β-catenin. Liu et al. (2005) report on 2-amino-4-[3,4-(methylenedioxy)benzyl-amino]-6-(3-methoxyphenyl)pyrimidine as an agonist of Wnt signaling.

Several important effects of the canonical Wnt pathway include cancers (alterations of Wnts, APC, axin, and TCFs are all associated with carcinogenesis), body axis specification (injection of Xenopus embryos with Wnt activators is involved in the development of a second body axis; Wnt is extensively involved in formation of the posterior nervous system and are released by tail “organizers”), and morphogenic signaling (Wnts produced from specific sites, such as the edge of the developing fly wing or the ventral edge of the neural tube of the developing vertebrate, are distributed throughout adjacent tissues in a gradient fashion; the Wnt pathway becomes activated to different degrees in cells of these tissues depending on how close they are to the production site, leading to subtle but crucial differences in the level of genes regulated by the Wnt pathway).

### III. Pyrvinium Compounds as Therapeutic Agents

A. Properties and Synthesis

The present invention relates to pyrvinium (U.S. Pat. No. 2,925,417), or salt, or an analog thereof that demonstrate potents anti-cancer activity in colon, breast and melanoma cancer cells. Pyrvinium has historically been used in the treatment of enterobiasis caused by Enterobius vermicularis (pinworm). However, pyrvinium has generally been replaced by other anthelmintics (e.g., mebendazole or pyrantel). It appears to prevent the parasite from utilizing exogenous carbohydrates.

Pyrvinium is shown here to inhibit cell growth and is therefore useful in the treatment of diseases of uncontrolled proliferation, such as cancer. Thus, the present invention provides pyrvinium or an analog thereof as a therapeutic agent for treating cancer in a subject, such as adrenocortical, hepatocellular, hepatoblastoma, malignant melanoma, ovarian, Wilm's tumor, Barrett's esophageal, bladder, breast, gastric, head & neck, lung cell, mesothelioma, and cervical cancers. Also provided are methods for treating Wnt-related non-cancer disease states.

B. Administration

To kill cells, induce cell cycle arrest, inhibit cell growth, inhibit metastasis, inhibit angiogenesis or otherwise reverse or reduce the malignant phenotype of melanoma cancer cells, using the methods and compositions of the present invention, one would generally contact a cell with the pyrvinium compound, or salts or analogs thereof. The terms “contacted” and “exposed,” when applied to a cell, are used herein to describe the process by which a therapeutic agent is delivered to a target cell or are placed in direct juxtaposition with the target cell. To achieve cell killing or stasis, the therapeutic agent is delivered to a cell in an amount effective to induce cell cycle arrest, inhibit cell growth and induce apoptosis in the cell.

Pyrvinium or a salt or an analog thereof as a therapeutic agent may be administered to a subject more than once and at intervals ranging from minutes to weeks. One would generally ensure that a significant period of time did not expire between the time of each delivery, such that the agent would still be able to exert an advantageous effect on the cell. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Administration of pyrvinium or a salt or an analog thereof to a subject may be by any method know in the art for delivery of a therapeutic agent to a subject. For example, such methods may include, but are not limited to, oral, nasal, intramuscular, or intraperitoneal administration. Methods of administration are disclosed in detail elsewhere in this application.

### IV. Assays for Identifying Wnt Pathway Modulators

The present invention comprises, in another embodiment, methods for identifying modulators of the Wnt pathway. These assays may comprise random screening of large libraries of candidate substances; alternatively, the assays may be used to focus on particular classes of compounds selected with an eye towards structural attributes that are believed to make them more likely to modulate the Wnt pathway.

To identify a modulator, one generally will determine Wnt signaling in the presence and absence of the candidate substance, a modulator defined as any substance that alters that signaling. For example, a method generally comprises:

(a) providing a Xenopus laevis egg extract comprising axin and β-catenin;

(b) contacting the extract with a candidate substance;

(c) assessing the degradation and/or stability of the axin and/or β-catenin,

wherein a change in the degradation and/or stability of axin and/or β-catenin, as compared to the degradation and/or stability of axin and/or β-catenin in the absence of said candidate substance, indicates that said candidate substance is a modulator of the Wnt pathway. Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

1. Modulators

As used herein the term “candidate substance” refers to any molecule that may potentially inhibit or enhance Wnt signaling. The candidate substance may be a protein or fragment thereof, a small molecule, or even a nucleic acid molecule. It may prove to be the case that the most useful pharmacological compounds will be compounds that are structurally related to compounds already identified, such as flavonoids generally, or pyrvinium in particular. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect in the Wnt pathway. Regardless of the type of inhibitor or activator identified by the present screening methods, the effect of the inhibition or activation is to alter Wnt signaling as compared to that observed in the absence of the candidate substance.

2. In Vitro/In Cyto Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces. Depending on the assay, culture of a cell may be required.

Of particular interest in the present invention is the use of a cell free system based on Xenopus laevis egg extracts. These extracts are very well known to those in the field. The African clawed frog (Xenopus laevis, also known as platanna) is a species of South African aquatic frog of the genus Xenopus. It is up to 12 cm long with a flattened head and body but no tongue. Its name derives from its three short claws on each of its hind feet, which it probably uses to stir up mud to hide it from predators. It is found throughout much of Europe, North America, South America, and Africa.

Xenopus oocytes provide an important expression system for various aspects of molecular biology. By injecting cDNA or cRNA into the developing oocyte, scientists can study the protein products in a controlled system. This allows rapid functional expression of manipulated cDNAs (or cRNA). One challenge of oocyte work is eliminating native proteins that might confound results, such as membrane channels native to the oocyte. Translation of proteins can be blocked or splicing of pre-mRNA can be modified by injection of m antisense oligos into the oocyte (for distribution throughout the embryo) or early embryo (for distribution only into daughter cells of the injected cell). The inventors here have advantageously utilized this system, in a cell free form, for assessing signaling through the Wnt pathway by virtue of examining the fate of axin and β-catenin.

3. In Vivo Assays

In vivo assays involve the use of various animal models, including transgenic animals that have been engineered to have specific defects, or to carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to Wnt signaling, but instead may look at a broader indication such as inhibition of tumor growth or metastasis, or induction of tumor cell death (apoptosis).

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, local or regional administration to a tumor via blood or lymph supply, or directly to a tumor site, e.g., intratumoral injection.

Determining the effectiveness of a compound in vivo may involve a variety of different criteria. Also, measuring toxicity and dose response can be performed in animals in a more meaningful fashion than in in vitro or in cyto assays.

### V. Combined Cancer Therapy

In the context of the present invention, it is contemplated that the pyrvinium compound, or salts or analogs thereof may be used in combination with a second therapeutic agent to more effectively treat a cancer. Additional therapeutic agents contemplated for use in combination with the pyrvinium compound, or salts or analogs thereof include, but are not limited to anticancer agents. Anticancer agents may include but are not limited to, radiotherapy, chemotherapy, gene therapy, hormonal therapy or immunotherapy that targets cancer/tumor cells.

To kill cells, induce cell-cycle arrest, inhibit cell growth, inhibit metastasis, inhibit angiogenesis or otherwise reverse or reduce the malignant phenotype of cancer cells, using the methods and compositions of the present invention, one would generally contact a cell with a pyrvinium compound, or salts or analogs thereof in combination with a second therapeutic agent. These compositions would be provided in a combined amount effective to kill or inhibit proliferation of the cell. This process may involve contacting the cells with pyrvinium, or salts or analogs thereof in combination with a second therapeutic agent or factor(s) at the same time. This may be achieved by contacting the cell with a single composition or pharmacological formulation that includes both agents, or by contacting the cell with two distinct compositions or formulations, at the same time, wherein one composition includes the pyrvinium or derivatives thereof and the other includes the second agent.

Alternatively, treatment with pyrvinium, or salts or analogs thereof may precede or follow the additional agent treatment by intervals ranging from minutes to weeks. In embodiments where the second agent is applied separately to the cell, one would generally ensure that a significant period of time did not expire between the time of each delivery, such that the agent would still be able to exert an advantageously combined effect on the cell. In such instances, it is contemplated that one would contact the cell with both modalities within about 12-24 hr of each other and, more preferably, within about 6-12 hr of each other, with a delay time of only about 12 hr being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either pyrvinium or salts, or analogs thereof in combination with a second therapeutic agent such as a anticancer agent or anticancer agent will be desired. Various combinations may be employed, where pyrvinium or salts or analogs thereof is “A” and the second therapeutic agent is “B”, as exemplified below:

| | | | | | |
|---|---|---|---|---|---|
| A/B/A | B/A/B | B/B/A | A/A/B | B/A/A | A/B/B |
| B/B/B/A | B/B/A/B | A/A/B/B | A/B/A/B | A/B/B/A | B/B/A/A |
| B/A/B/A | B/A/A/B | B/B/B/A | A/A/A/B | B/A/A/A | A/B/A/A |
| A/A/B/A | A/B/B/B | B/A/B/B | B/B/A/B | | |

Other combinations are contemplated. Again, to achieve cell killing by the induction of apoptosis, both agents may be delivered to a cell in a combined amount effective to kill the cell.

A. Chemotherapeutic Agents

The present invention also contemplates the use of chemotherapeutic agents in combination with BMS-345541 or an analog thereof in the treatment of melanoma cancer. Examples of such chemotherapeutic agents may include, but are not limited to, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, gemcitabien, navelbine, farnesyl-protein transferase inhibitors, transplatinum, 5-fluorouracil and methotrexate, or any analog or derivative variant of the foregoing.

B. Radiotherapeutic Agents

Radiotherapeutic agents may also be use in combination with the compounds of the present invention in treating a melanoma cancer. Such factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapeutic Agents

Immunotherapeutics may also be employed in the present invention in combination with pyrvinium or salts or analogs thereof in treating melanoma cancer. Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, mda-7, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

E. Regulators of Programmed Cell Death

Apoptosis, or programmed cell death, is an essential process in cancer therapy (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Members of the Bcl-2 that function to promote cell death such as, Bax, Bak, Bik, Bim, Bid, Bad and Harakiri, are contemplated for use in combination with BMS-345541 or an analog thereof in treating melanoma cancer.

F. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increased intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

G. Surgery

It is further contemplated that a surgical procedure may be employed in the present invention. Approximately 60% of persons with cancer will undergo surgery of some type, which includes preventative, diagnostic or staging, curative and palliative surgery. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with a second anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### VI. Formulations and Routes for Administration

Where clinical applications are contemplated, it will be necessary to prepare pharmaceutical compositions of pyrvinium or salts or analogs thereof, or any additional therapeutic agent disclosed herein in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention in an effective amount may be dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The composition(s) of the present invention may be delivered orally, nasally, intramuscularly, intratumorally, intraperitoneally. In some embodiments, local or regional delivery of pyrvinium or salts or analogs thereof alone, or in combination with a second therapeutic agent, to a patient with cancer or pre-cancer conditions will be a very efficient method of delivery to counteract the clinical disease. Similarly, chemo- or radiotherapy may be directed to a particular, affected region of the subject's body. Regional chemotherapy typically involves targeting anticancer agents to the region of the body where the cancer cells or tumor are located. Other examples of delivery of the compounds of the present invention that may be employed include intra-arterial, intracavity, intravesical, intrathecal, intrapleural, and intraperitoneal routes.

Intra-arterial administration is achieved using a catheter that is inserted into an artery to an organ or to an extremity. Typically, a pump is attached to the catheter. Intracavity administration describes when chemotherapeutic drugs are introduced directly into a body cavity such as intravesical (into the bladder), peritoneal (abdominal) cavity, or pleural (chest) cavity. Agents can be given directly via catheter. Intravesical chemotherapy involves a urinary catheter to provide drugs to the bladder, and is thus useful for the treatment of bladder cancer. Intrapleural administration is accomplished using large and small chest catheters, while a Tenkhoff catheter (a catheter specially designed for removing or adding large amounts of fluid from or into the peritoneum) or a catheter with an implanted port is used for intraperitoneal chemotherapy. Abdomen cancer may be treated this way. Because most drugs do not penetrate the blood/brain barrier, intrathecal chemotherapy is used to reach cancer cells in the central nervous system.

Alternatively, systemic delivery of the chemotherapeutic drugs may be appropriate in certain circumstances, for example, where extensive metastasis has occurred. Intravenous therapy can be implemented in a number of ways, such as by peripheral access or through a vascular access device (VAD). A VAD is a device that includes a catheter, which is placed into a large vein in the arm, chest, or neck. It can be used to administer several drugs simultaneously, for long-term treatment, for continuous infusion, and for drugs that are vesicants, which may produce serious injury to skin or muscle. Various types of vascular access devices are available.

The active compositions of the present invention may include classic pharmaceutical preparations. Administration of these compositions according to the present invention will be via any common route so long as the target tissue is available via that route. This includes but is not limited to, oral, nasal, or buccal routes. Alternatively, administration may be by orthotopic, intradermal, subcutaneous, intramuscular, intraperitoneal or intravenous injection. Such compositions would normally be administered as pharmaceutically acceptable compositions, described supra. The drugs and agents also may be administered parenterally or intraperitoneally. The term “parenteral” is generally used to refer to drugs given intravenously, intramuscularly, or subcutaneously.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions of the present invention may be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH, exact concentration of the various components, and the pharmaceutical composition are adjusted according to well known parameters. Suitable excipients for formulation with pyrvinium or salts or analogs thereof include croscarmellose sodium, hydroxypropyl methylcellulose, iron oxides synthetic), magnesium stearate, microcrystalline cellulose, polyethylene glycol 400, polysorbate 80, povidone, silicon dioxide, titanium dioxide, and water (purified).

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent(s) of the present invention is determined based on the intended goal, for example (i) inhibition of tumor cell proliferation or (ii) elimination of tumor cells. The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

### VII. Therapeutically Effective Amounts of Pyrvinium Compositions

A therapeutically effective amount of pyrvinium, or salts, or analogs thereof alone, or in combination with a second therapeutic agent such as an anticancer agent as a treatment varies depending upon the host treated and the particular mode of administration. In one embodiment of the invention the dose range of the pyrvinium or salts or analogs thereof alone, or in combination with a second agent used will be about 0.5 mg/kg body weight to about 500 mg/kg body weight. The term “body weight” is applicable when an animal is being treated. When isolated cells are being treated, “body weight” as used herein should read to mean “total cell weight”. The term “total weight” may be used to apply to both isolated cell and animal treatment. All concentrations and treatment levels are expressed as “body weight” or simply “kg” in this application are also considered to cover the analogous “total cell weight” and “total weight” concentrations. However, those of skill will recognize the utility of a variety of dosage range, for example, 1 mg/kg body weight to 450 mg/kg body weight, 2 mg/kg body weight to 400 mg/kg body weight, 3 mg/kg body weight to 350 mg/kg body weight, 4 mg/kg body weight to 300 mg/kg body weight, 5 mg/kg body weight to 250 mg/kg body weight, 6 mg/kg body weight to 200 mg/kg body weight, 7 mg/kg body weight to 150 mg/kg body weight, 8 mg/kg body weight to 100 mg/kg body weight, or 9 mg/kg body weight to 50 mg/kg body weight. Further, those of skill will recognize that a variety of different dosage levels will be of use, for example, 1 mg/kg, 2 mg/kg, 3 mg/kg, 4 mg/kg, 5 mg/kg, 7.5 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 120 mg/kg, 140 mg/kg, 150 mg/kg, 160 mg/kg, 180 mg/kg, 200 mg/kg, 225 mg/kg, 250 mg/kg, 275 mg/kg, 300 mg/kg, 325 mg/kg, 350 mg/kg, 375 mg/kg, 400 mg/kg, 450 mg/kg, 500 mg/kg, 550 mg/kg, 600 mg/kg, 700 mg/kg, 750 mg/kg, 800 mg/kg, 900 mg/kg, 1000 mg/kg, 1250 mg/kg, 1500 mg/kg, 1750 mg/kg, 2000 mg/kg, 2500 mg/kg, and/or 3000 mg/kg. Of course, all of these dosages are exemplary, and any dosage in-between these points is also expected to be of use in the invention. Any of the above dosage ranges or dosage levels may be employed for pyrvinium, or salts or analogs thereof in combination with a second therapeutic agent.

“Therapeutically effective amounts” are those amounts effective to produce beneficial results, particularly with respect to cancer treatment, in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

As is well known in the art, a specific dose level of active compounds such as pyrvinium or salts or analogs thereof alone, or in combination with a second therapeutic agent, for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

In some embodiments, pyrvinium or salts or analogs thereof alone, or in combination with a second therapeutic agent will be administered. When a second therapeutic agent is administered, as long as the dose of the second therapeutic agent does not exceed previously quoted toxicity levels, the effective amounts of the second therapeutic agents may simply be defined as those amounts effective to reduce the cancer growth when administered to an animal in combination with the pyrvinium or salts or analogs thereof. This may be easily determined by monitoring the animal or patient and measuring those physical and biochemical parameters of health and disease that are indicative of the success of a given treatment. Such methods are routine in animal testing and clinical practice.

In some embodiments of the present invention chemotherapy may be administered, as is typical, in regular cycles. A cycle may involve one dose, after which several days or weeks without treatment ensues for normal tissues to recover from the drug's side effects. Doses may be given several days in a row, or every other day for several days, followed by a period of rest. If more than one drug is used, the treatment plan will specify how often and exactly when each drug should be given. The number of cycles a person receives may be determined before treatment starts (based on the type and stage of cancer) or may be flexible, in order to take into account how quickly the tumor is shrinking. Certain serious side effects may also require doctors to adjust chemotherapy plans to allow the patient time to recover.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### A. Materials & Methods

Screen format. The inventors conducted, in Xenopus egg extracts, a chemical screen of the Wnt pathway utilizing in vitro reconstitution of the essential signal transduction events. Addition of β-catenin-luciferase and Axin-Renilla-luciferase to extracts allowed us to simultaneously test the ability of drug libraries to either activate or inhibit Wnt signaling in vitro. Recombinant intracellular domain of the Wnt receptor LRP6 was added to extracts to stabilize β-catenin and stimulate Axin degradation, thereby “activating” the Wnt pathway. Small molecules were screened to identify compounds that regulate β-catenin and Axin turnover in extract.

A master mix included the following:
100 ml low speed Xenopus extract (protease inhibitors and cytochalasin added previously upon preparation)
5 ml Energy Regeneration system (20× stock)
1 ml human β-catenin-luciferase (firefly) expressed in SP6 high yield wheat germ lysate (Promega)
1.5 ml mouse Axin-Renilla-luciferase expressed in SP6 high yield wheat germ (Promega).
6 ml recombinant LRP6-intracellular domain (0.5 mg/ml)
   This was incubated for 5 min at 4° C., aliquotted at 5 μl/well into standard volume 384-well plate (Corning 3705) using 24 channel repeat Pipetman® in a cold room. Pin-transfer of 100 nl of compounds was performed at room temperature. Compound stocks ranged from 2 mM to 10 mM in DMSO (2% final DMSO concentration, 40-200 μm compound final concentration). Plates were sealed manually with adhesive from mailing labels (Staples) and incubated 4 hr at room temperature. Twenty μl of Reagent 1 was aliquotted (Promega Dual Glo, 1:1 in water) using Well Mate. Plates were read using EnVision 2. Twenty μl of Reagent 2 was aliquotted (Promega Dual Glo, 1:1 in water) using Well Mate. Again, plates were read using EnVision 2.

Data analysis. A large data set was generated by screening for both β-catenin and Axin signal. The size of this data seat was reduced by eliminating wells that had either a β-catenin or Axin signal in the middle-third of the entire range of signal, thus eliminating compounds that did not significantly change the β-catenin/axin signals. Next, the Axin value was divided by the β-catenin value, providing a ratio of the two signals for each well. Large values represent putative Wnt inhibitors (e.g., large Axin value divided by a small β-catenin value), and small values represent putative Wnt activators (e.g., snall Axin value divided by a large β-catenin value).

Radiolabeled Axin degradation. S³⁵-labeled Axin was generated using rabbit reticulocyte lysate according to standard protocols. Radiolabeled Axin (0.1 μl) and pyrvinium pamoate (0.2 μl of 50 mM stock) was added to Xenopus egg extract (10 μl) on ice. The extract was warmed to 25° C., and 1 μl was removed and added to hot sample buffer at indicated time points. Samples were analyzed by SDS-PAGE and autoradiography.

β-catenin Western blot and RT-PCR. HEK 293 cells were seeded at 90% confluency in a 6-well dish. After cells adhered to the wells, Wnt3a-conditioned medium minus or plus Pryvinium pamoate was added (4 ml total). Cells were collected for analysis after 24 hr. For Western blot, cells were lysed in hypotonic buffer, spun at 16,000×g, and the supernatant retained for analysis. Lysates were probed for β-catenin and actin (loading control) using to standard techniques.

For RT-PCR, total messenger RNA was prepared using RNA Stat-60 (Ambion). PCR was performed using Ready-To-Go™ RT-PCR Beads (GE Healthcare) according to manufacturer's protocol. Amplification was carried out at 94° C. for initial denaturation followed by cycles of 94° C. for 20 s, 60° C. for 20 s, and 72° C. for 30 s. Products were electrophoresed on 2% agarose gels and detected by EtBr staining. Primer sequences are as follows:

| | |
|---|---|
| Wnt targets | |
| DKK1-FW: | |
| 5′-TCCCCTGTGATTGCAGTAAA-3′ | (SEQ I NO: 1) |
| DKK1-REV: | |
| 5′-TCCAAGAGATCCTTGCGTTC-3′ | (SEQ I NO: 2) |
| Axin2-FW: | |
| 5′-AGTGTGAGGTCCACGGAAAC-3′ | (SEQ I NO: 3) |
| Axin2-REV: | |
| 5′-CTTCACACTGCGATGCATTT-3′ | (SEQ I NO: 4) |
| Control genes | |
| GAPDH-FW: | |
| 5′-ACAGTCA GCCGCATCTTCTT-3′ | (SEQ I NO: 5) |
| GAPDH-REV: | |
| 5′-GACAAGCTTCCCGTTCTCAG-3′ | (SEQ I NO: 6) |

Topflash reporter assay. HEK 293 cells stably expressing 8× Supertopflash were seeded in quadruplicate at 15,000 cells/well/100 μl of medium in 96 well-plate. After cells adhered, 1 μl of 100× pyrvinium pamoate was added for one hr. Next, 100 μl of Wnt3a-conditioned media and 1 μl of 100× pyrvinium pamoate were added and cells incubated for 8 hr. Cells were lysed in passive lysis buffer (Promega), and luciferase was measured according to Promega's Steady-Glo protocol.

Xenopus embryo experiments. Xenopus embryos were fertilized in vitro, dejellied in 2% cysteine (pH 7.8), and cultured in 10% MMR (0.1 M NaCl, 2.0 mM KCl, 1 mM MgSO4, 2 mM CaCl₂, 5 mM Hepes, pH 7.8, 0.1 mM EDTA). For microinjections, embryos were kept in 3% Ficoll in 0.2×MMR. In vitro-transcribed, capped mRNA was synthesized using the mMessage mMachine Kit (Ambion). For the axis duplication assay, Xwnt8 mRNA (0.5 pg) was co-injected with 100 μM of pyrvinium pamoate (2.5 nl of 10 mM stock solution in DMSO) into the equatorial region of one of the two ventral blastomeres at the four-cell stage.

Cancer cell growth, viability, and apoptosis assays. HCT116, SW620, SW480, MDA-MB-231 cancer lines were maintained according to ATCC guidelines. For cell growth and viability assays, cells were seeded at 2000 cells/100 μl/well in 96-well plates. Cell number was measured at different time points using CellTiter-Glo® Luminescent Cell Viability Assay (Promega). For apoptosis assays, cells were seeded at 5000 cells/100 μl/well in 96-well plates. Drug was added for 24 hr and Caspase-3 and -7 activities were measured using Apo-ONE® Homogeneous Caspase-3/7 Assay (Promega).

### B. Results

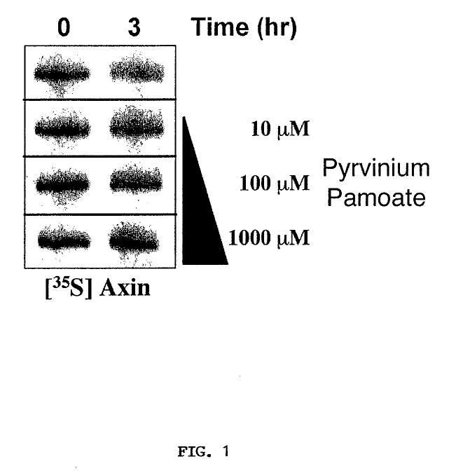

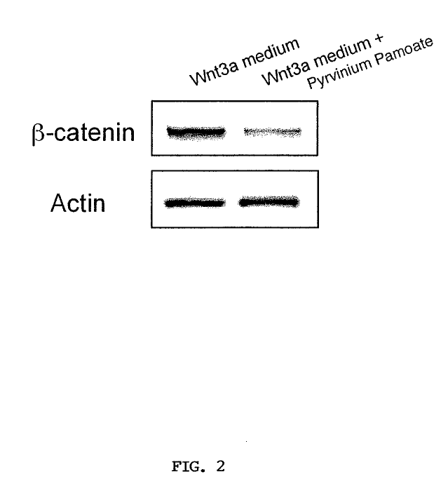

Pyrvinium pamoate stabilizes Axin and destabilizes β-catenin. One of the strongest “hits” from an initial small molecule screen was the anthelminthic agent, pyrvinium, which enhanced Axin-Renilla luciferase and decreased the β-catenin-firefly luciferase signal in the screen described above. To test whether this effect was a consequence of regulation of the Wnt pathway as opposed to a non-specific effect on firefly and Renilla luciferases, the inventors tested pyrvinium's effect on the rate of degradation of full-length, radiolabeled Axin (that was not fused to Renilla luciferase) in Xenopus extracts. Indeed, we found that pyrvinium blocks Axin degradation in a dose-dependent manner (FIG. 1). Given that Axin is the limiting component of the β-catenin destruction complex, they predicted that stabilization of Axin by small molecules would block Wnt signal transduction by elevating levels of complete β-catenin destruction complexes, thereby decreasing levels of β-catenin in the cell. To show pyrvinium has the predicted effect on β-catenin levels in intact cells, the inventors tested this drug's effect on HEK 293 cultured cells that are wild-type for Wnt pathway components. In accordance with the reversal of Wnt signaling, HEK 293 cells that were stimulated with Wnt3a-conditioned medium and pyrvinium for 24 hr had decreased levels of cytosolic β-catenin (FIG. 2). Together, the stabilization of Axin and the destabilization of beta-catenin by pyrvinium validate the ability of our screening approach to accurately identify compounds that regulate Axin/β-catenin stability. In addition, these results exemplify pyrvinium's ability to reverse the key molecular events following Wnt ligand stimulation.

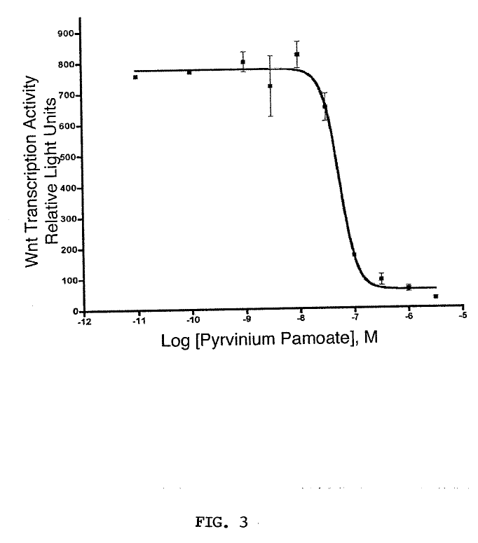

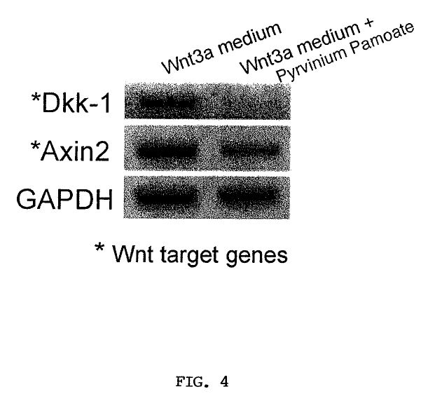

Pyrvinium pamoate blocks Wnt-mediated gene transcription. Upon Wnt stimulation, levels of β-catenin rise followed by its translocation to the cell nucleus and subsequent interaction with the transcription co-factor TCF/LEF. This event recruits transcription machinery to a large set of genes involved in cellular processes such as proliferation and growth. Activation of these genes represents the major downstream effect of Wnt signaling. To test if pyrvinium can blocks activation of these genes, the inventors performed Topflash assays in HEK 293 cells. This assay uses an exogenous luciferase gene under the control of TCF promoter elements as a measure of active β-catenin-mediated gene transcription. A dose response curve of pyrvinium on HEK 293 cells stimulated with Wnt3a demonstrated a dose-dependent inhibition of Wnt-mediated transcription with an EC₅₀ below 50 nM (FIG. 3). Likewise, they tested pyrvinium's effects on endogenous gene targets, Axin2 and Dkk-1, and found that pyrvinium potently decreases mRNA levels of both of these Wnt targets (FIG. 4)

Pyrvinium pamoate blocks Wnt signaling in vivo. The inventors have shown Wnt-inhibiting activity of pyrvinium biochemically in Xenopus egg extracts as well as in mammalian cell culture. To test this compound in the context of a whole organism, they used Xenopus embryos as a model system. A common in vivo assay for early Wnt signaling in the embryo is the induction of a secondary axis by injection of pathway activators into the ventral blastomeres at the 4- or 8-cell stage. Embryos injected with Wnt-8 develop a full secondary axis. The inventors found that co-injection of Wnt8 with pyrvinium result in complete block of secondary axis induction. Importantly, pyrvinium appears to specifically affect axis formation, a Wnt-regulated process, and does not have cytotoxic effects on the whole embryo. This result exemplifies the specificity of pyrvinium for the Wnt pathway as oppose to other developmental signaling cues.

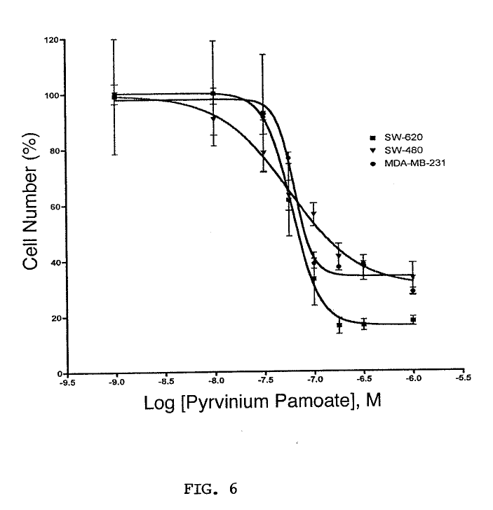

Pyrvinium blocks cancer cell proliferation and induces apoptosis. As stated above, elevated Wnt/β-catenin signaling drives numerous types of cancers. To show the effectiveness of pyrvinium on cancer, we conducted cell viability dose-response curves on a number of cell lines with well-characterized constitutive Wnt activity. Colorectal cancers lines SW620 and SW480 harbor APC mutations. The metastatic breast cancer line MDA-MB-2321 has no mutation in Wnt components but secretes high levels of Wnts that signal in an autocrine fashion to drive proliferation. We incubated these cells lines with varying doses of pyrvinium and measured cell viability after 48 hr. All lines show a strong dose response with an EC₅₀ below 50 nM, similar to the EC₅₀ for Wnt inhibition (FIG. 6). These results demonstrate pyrvnium's effect on cancer cell lines with autocrine Wnt signaling or mutant Wnt pathway components.

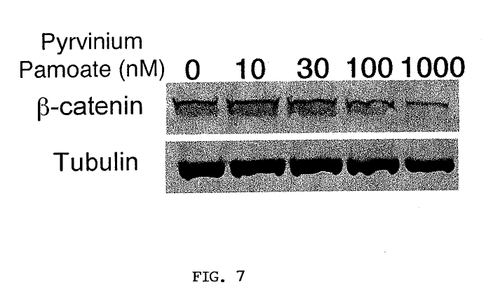

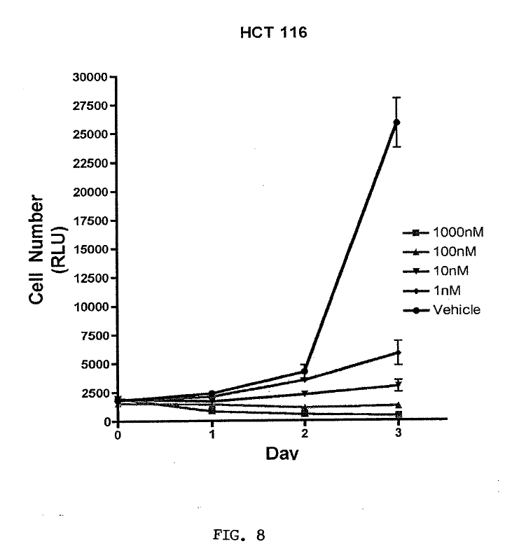

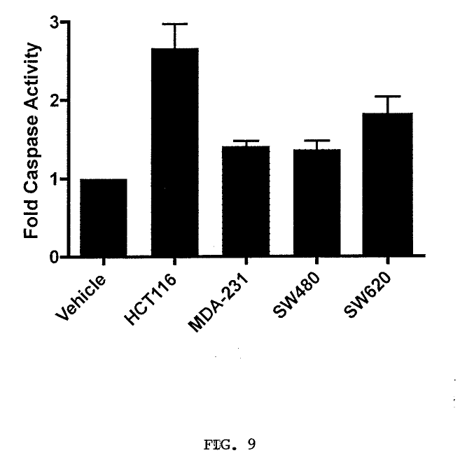

The inventors tested the effects of pyrvinium on an additional colorectal cancer line, HCT116. This line contains a mutation on one allele of β-catenin that prevents its phosphorylation by the destruction complex. They first analyzed the effect of pyrvinium on cytoplasmic levels of β-catenin. Interestingly, pyrvinium decreased levels of β-catenin but not completely (FIG. 7). The remaining β-catenin represents the non-degradable form of β-catenin. They next tested if pyrvinim could block proliferation of HCT116 cells despite this mutation in β-catenin. Elevation of Axin levels can sequester β-catenin in the cytoplasm, keeping it out of the nucleus and preventing its interaction with TCF. Thus, pyrvinium should have similar effects on growth of HCT116 cells as observed for other cell lines despite the non-degradable β-catenin allele. The inventors conducted a growth curve assay of HCT116 cells treated with pyrvinium for 72 hr (FIG. 8), and its effects were equally a potent as in the other cell lines. Significantly, this result demonstrates pyrvinium's potency even when in a cell line that is mutated for a key downstream effector of Wnt signaling, β-catenin. From this growth curve, they noticed that cells were dying at higher doses of pyrvinium. To test whether cells were dying via apoptosis, they measured the activity of caspase-3 and -7 after 24 hr of pyrvinium treatment (FIG. 9) and found it was elevated for all the cancer cell lines tested, consistent with stimulation of apoptosis by pyrvinium.

### Example 2

### A. Materials & Methods

For neurosphere assays, primary cultures from three human brain tumors were plated in tumor stem cell medium (TSM) (1), a chemically defined serum-free neural stem cell medium, containing EGF and bFGF. Tumor cells were plated at a density of 200 cells/well and the number of spheres assessed 48 hours later in the presence of vehicle or 100 nM pyrvinium.

For apoptosis assays, SW620 cells were seeded at 5000 cells/100 ul/well in 96 well plates. Pyrvinium and/or 5FU (Sigma) was added after attachment and incubated at 37° C. and 5% CO₂. After 24 hours, phase contrast images were obtained followed by measurement of Caspase 3 and 7 by Apo-ONE® Homogeneous Caspase-3/7 Assay (Promega) according to manufactures instructions.

### B. Results

The effect of pyrvinium on multipotent self-renewing tumor cells (cancer stem cells) was tested in a neurosphere assay using primary glioblastoma tumor cells from three different individuals. Although the ability to form neurospheres varies between each cell line, the ability to form neurosphere within each line is very reproducible. As shown in the figure, the vehicle had no effect on neurosphere formation for all three lines. However, pyrvinium at 100 nM completely abrogated the ability of all three lines to form neurospheres.

The ability of pyrvinium to target cancer cells is further demonstrated by the fact that in the presence of pyrvinium, 5-FU-induced apoptosis of a colorectal cancer cell line is greatly enhanced in a cell line (SW620) that is normally resistant to apoptosis.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 2,925,417
Arap et al., Cancer Res., 55(6):1351-1354, 1995.
Bakhshi et al., Cell, 41(3):899-906, 1985.
Caldas et al., Nat. Genet., 8(1):27-32, 1994.
Cheng et al., Cancer Res., 54(21):5547-5551, 1994.
Cleary and Sklar, Proc. Natl. Acad. Sci. USA, (21):7439-7443, 1985.
Cleary et al., J. Exp. Med., 164(1):315-320, 1986.
Hollstein et al., Science, 253(5015):49-53, 1991.
Hussussian et al., Nat. Genet., 8(1):15-21, 1994.
Kamb et al., Nat. Genet., 8(1):23-26, 1994.
Kerr et al., Br. J. Cancer, 26(4):239-257, 1972.
Liu et al., Am. J. Gastroenterol., 100:792-796, 2005.
Mori et al., Cancer Res., 54(13):3396-3397, 1994.
Nobri et al., Nature (London), 368:753-756, 1995.
Okamoto et al., Proc. Natl. Acad. Sci. USA, 91(23):11045-11049, 1994.
Orlow et al., Int. J. Oncol., 15(1):17-24, 1994.
Reynolds & Weiss, Science 255: 1707-1710, 1992.
Serrano et al., Nature, 366:704-707, 1993.
Serrano et al., Science, 267(5195):249-252, 1995.
Tsujimoto and Croce, Proc. Natl. Acad. Sci. USA, 83(14):5214-5218, 1986.
Tsujimoto et al., Science, 228(4706):1440-1443, 1985.
Weinberg, Science, 254(5035):1138-1146, 1991.

What is claimed is:

1. A method of identifying modulators for the Wnt pathway comprising:
(a) providing a Xenopus laevis egg extract comprising axin and β-catenin;
(b) contacting said extract with a candidate substance;
(c) assessing the degradation and/or stability of said axin and/or β-catenin,
wherein a change in the degradation and/or stability of axin and/or β-catenin, as compared to the degradation and/or stability of axin and/or β-catenin in the absence of said candidate substance, indicates that said candidate substance is a modulator of the Wnt pathway.

2. The method of claim 1, wherein said axin and/or β-catenin molecules are labeled.

3. The method of claim 2, wherein said axin and/or β-catenin molecules are labeled by fusion with a fluorescent protein.

4. The method of claim 3, wherein said fluorescent protein is selected from luciferase, GPF, CFP, YFP, or ECPF.

5. The method of claim 2, wherein said axin and/or β-catenin molecules are labeled with a radioactive label or a dye.

6. The method of claim 1, wherein said modulator increases degradation of β-catenin and increases stability of axin, and said modulator is an inhibitor of the Wnt pathway.

7. The method of claim 1, wherein said modulator increases degradation of axin and increases stability of β-catenin, and said modulator is an activator of the Wnt pathway.

8. The method of claim 1, wherein said candidate substance is a organopharmaceutical drug.

9. The method of claim 1, wherein said candidate substance is an oligonucleotide or polynucleotide.

10. The method of claim 1, wherein said candidate substance is a peptide or polypeptide.

11. A method of inhibiting a cancer cell selected from the group consisting of an adrenocortical cancer cell, a hepatocellular cancer cell, a hepatoblastoma cell, a malignant melanoma cell, a ovarian cancer cell, a Wilm's tumor cell, a Barrett's esophageal cancer cell, a bladder cancer cell, a breast cancer cell, a gastric cancer cell, a head & neck cancer cell, a lung cancer cell, a mesothelioma cell, a cervical cancer cell, a glioblastoma cell, a uterine cancer cell, a myeloid leukemia cancer cell, a lymphoid leukemia cancer cell, a pilometricoma cancer cell, a medulloblastoma cancer cell, and a familial adenomatous polyposis cancer cell comprising contacting said cancer cell with a composition comprising pyrvinium.

12. The method of claim 11, wherein said cancer cell is located in a subject.

13. The method of claim 12, wherein said subject is a human.

14. The method of claim 12, wherein said cancer cell is metastatic.

15. The method of claim 12, wherein said cancer cell is multi-drug resistant.

16. The method of claim 12, wherein said cancer cell is recurrent.

17. The method of claim 11, wherein said cancer cell has a mutation in APC, β-catenin, and/or axin.

18. The method of claim 11, further comprising subjecting said cancer cell with a second anti-cancer therapy.

19. The method of claim 18, wherein said second anti-cancer therapy is chemotherapy, radiotherapy, gene therapy, immunotherapy, hormone therapy, toxin therapy, protein/peptide therapy, or surgery.

20. The method of claim 18, wherein said second anti-cancer therapy is given prior to said pyrvinium composition.

21. The method of claim 18, wherein said second anti-cancer therapy is given after said pyrvinium composition.

22. The method of claim 18, wherein said second anti-cancer therapy is given at the same time as said pyrvinium composition.

23. The method of claim 11, wherein said pyrvinium composition is contacted with said cancer cell more than once.

24. The method of claim 11, wherein inhibiting comprises reducing cancer cell growth.

25. The method of claim 11, wherein inhibiting comprises killing said cancer cell.

26. The method of claim 12, wherein said pyrvinium composition is administered orally, intravenously, intratumorally, into tumor vasculature, or regional to said tumor.

27. A method of treating a non-cancer disease state have a Wnt signaling abnormality comprising administering to a subject in need thereof a composition comprising pyrvinium.

28. The method of claim 27, wherein said subject is a human.

29. The method of claim 27, further comprising subjecting said cancer cell with a second therapy.

30. The method of claim 29, wherein said second therapy is given prior to said pyrvinium composition.

31. The method of claim 29, wherein said second therapy is given after said pyrvinium composition.

32. The method of claim 29, wherein said second therapy is given at the same time as said pyrvinium composition.

33. The method of claim 27, wherein said pyrvinium composition is administered more than once.

34. The method of claim 27, wherein said non-cancer disease state is autism, rheumatoid arthritis, schizophrenia, increased bone density, cardiac hypertrophy, Alzheimer's Disease, coronary artery disease, obesity, osteoporosis, familial exudative vitreoretinopathy, type II diabetes, pulmonary fibrosis, inflammation, or wound healing.

35. A method of stimulating a stem cell comprising contacting said stem cell with a composition comprising pyrvinium.

36. A method of inhibiting a well-vascularized tumor in a subject comprising administering to said subject a composition comprising pyrvinium.


SEQUENCE LISTING (6 sequences)

SEQ ID NO: 1 - DNA, 20 bp, Artificial
  Artificial primer
tcccctgtgattgcagtaaa

SEQ ID NO: 2 - DNA, 20 bp, Artificial
  Artificial primer
tccaagagatccttgcgttc

SEQ ID NO: 3 - DNA, 20 bp, Artificial
  Artificial primer
agtgtgaggtccacggaaac

SEQ ID NO: 4 - DNA, 20 bp, Artificial
  Artificial primer
cttcacactgcgatgcattt

SEQ ID NO: 5 - DNA, 20 bp, Artificial
  Artificial primer
acagtcagccgcatcttctt

SEQ ID NO: 6 - DNA, 20 bp, Artificial
  Artificial primer
gacaagcttcccgttctcag